(19) United States
(12) Patent Application Publication
(10) Pub. No.: US 2020/0011778 A1
(43) Pub. Date: Jan. 9, 2020

(54) ELECTROCHEMICAL OSMOLARITY OR OSMOLALITY SENSOR FOR CLINICAL ASSESSMENT

(71) Applicants: ARIZONA BOARD OF REGENTS ON BEHALF OF ARIZONA STATE UNIVERSITY, Scottsdale, AZ (US); ADVANCED TEAR DIAGNOSTICS, Birmingham, AL (US)

(72) Inventors: Mackenzie Honikel, Chandler, AZ (US); Chi Lin, Tempe, AZ (US); Andrew Penman, Birmingham, AL (US); Brittney Cardinell, Chandler, AZ (US); Jeffrey LaBelle, Tempe, AZ (US); Pierce Youngbar, Birmingham, AL (US); Marcus Smith, Birmingham, AL (US)

(21) Appl. No.: 16/482,661

(22) PCT Filed: Feb. 7, 2018
(86) PCT No.: PCT/US2018/017158

§ 371 Date: Jul. 31, 2019

Related U.S. Application Data
(60) Provisional application No. 62/455,913, filed on Feb. 7, 2017.

(51) Int. Cl. G01N 13/04; G01N 33/487; G01N 33/493; G01N 33/49

(52) U.S. Cl. CPC G01N 13/04; G01N 33/49; G01N 33/493; G01N 33/48707

(57) ABSTRACT

Osmolality and osmolality sensors and methods utilizing electrochemical impedance to detect changes in impedance to varying salinity concentrations. By way of example, the impedance reported at the specified frequency varies logarithmically with the concentration of sodium chloride subject to the sensor surface. Measurements obtained by the sensors and methods herein are utilized, for example, to differentiate between the clinical stages of dry eye disease (290-316 mOsm/L) to complement the current diagnostic procedures. Blood serum, urinalysis, and saliva also may be tested and the corresponding osmolarity or osmolality level evaluated for indications of a disease or condition.

### CROSS-REFERENCE TO RELATED APPLICATIONS

This application claims priority to U.S. Provisional Patent Application No. 62/455,913 filed on Feb. 7, 2017, the disclosure of which is incorporated herein in its entirety by reference.

### FIELD OF THE INVENTION

This disclosure is related to condition and disease detection tools and related methods involving measurement of electrochemical osmolarity or osmolality.

### BACKGROUND OF THE INVENTION

Dry eye is often qualitatively diagnosed through mechanisms linked to tear production volume (Schirmer Test) and patient symptoms (irritation and inflammation). Tear composition plays an important role in the identification of the severity of this disease. The development and implementation of a device to quantify the salinity of tear fluid will complement the current practices of ophthalmologists and lead to improved treatment.

Moreover, many other conditions may be better assessed through the measurement of osmolarity or osmolality, such as dehydration based on serum osmolality. Accordingly, devices and methods that quantify the salinity of body fluid samples could find widespread application.

### SUMMARY OF THE INVENTION

Some embodiments herein relate to apparatus and methods for dry eye detection and diagnosis, as well as other ocular diseases, through the electrochemical impedance measurement of tear fluid osmolarity. For example, tear fluid can be drawn to a custom electrode from the eye using FDA approved filter paper. A range of tear fluid osmolarity associated with dry eye can be detected in the tear fluid using Electrochemical Impedance Spectroscopy (EIS) in a handheld point-of-care device. Accordingly, the embodiments herein can help improve the quality of life and the management of ocular conditions such as dry eye by providing more accurate information for medical assessment and treatment.

Other embodiments relate to measurement of osmolarity or osmolality in various bodily fluids for the assessment of conditions, such as dehydration, or indications of disease.

### BRIEF DESCRIPTION OF THE DRAWINGS

FIG. 1. Gold Disk Electrode Data. Current data represents sensor functionality on a benchtop system. The complex impedance can be correlated to solution (Distilled water and NaCl only) salinity at its optimal frequency (117.2 Hz). There is low relative standard deviation within the clinical range of interest.

FIG. 2. Screen Printed Electrode. The device demonstrates feasibility and proof of concept on screen printed electrodes, analogous to a final test strip design to be used in a clinical setting.

FIG. 3. Screen Printed Electrode Data. The screen printed gold sensors demonstrate a larger change in impedance with changing NaCl concentrations, thus giving the system an increased resolution. This represents the functional range of the system (3-30 mg/mL).

FIG. 4. Clinically relevant concentration ranges are shown.

FIGS. 5a-5b. Form-factor. Some embodiments are designed to fit comfortably in the hand and to mimic currently available products such as the Tono-pen. At the round tip for a handheld device, a disposable drum containing the test strip can be easily placed and discarded.

FIG. 6. This figure depicts another view of the embodiment of FIGS. 5a-5b.

FIG. 7. Sensor Strip—Fluid capture test strip concept: PVC or similar substrate; screen-printed electrode leads (incl. dried reagents and protein); filter paper to absorb tear fluid; shape and dimensions of filter paper to be determined based on absorption tests (˜1.75×1.75 mm); dimensions of 3-lead electrode to be determined based on filter paper dimensions; electrode materials: carbon conductive ink, silver chloride ink, novel mesoporous carbon ink and glue; mesoporous ink facilitates electrochemical measurement and contains protein to detect the molecule of interest.

FIGS. 8a-b. Sensor Strip—The sensor may consist of 4 layers of screen print inks, each with its own stencil. The complete sensor is shown (right) with a close view of the tip, where the filter paper will interface.

FIGS. 9a-d. Sensor Strip—The four layers of ink are shown as separate stencil designs as they would be printed, the first layer being carbon, then Ag/AgCl, etc.

FIGS. 10a-b. Saturation test—Determining actual tear fluid volume captured and reproducibility. 4 filter paper sizes were measured (n=5) to determine the amount of tear fluid each size can absorb when exposed to a 64 pool of tear fluid.

### DETAILED DESCRIPTION OF THE INVENTION

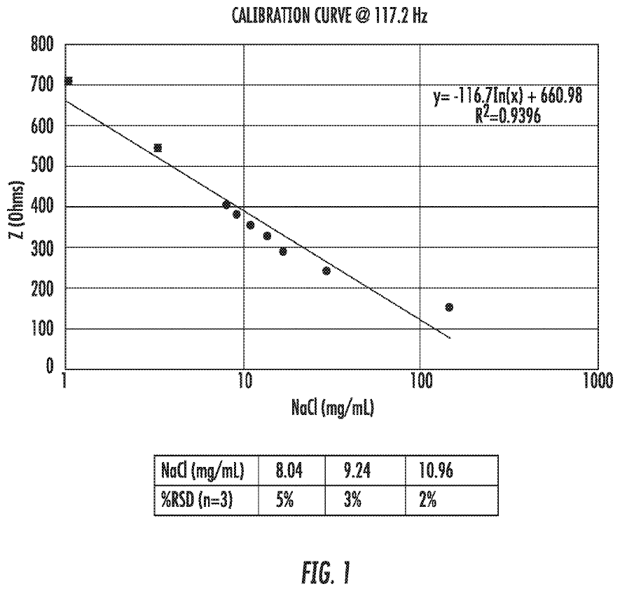

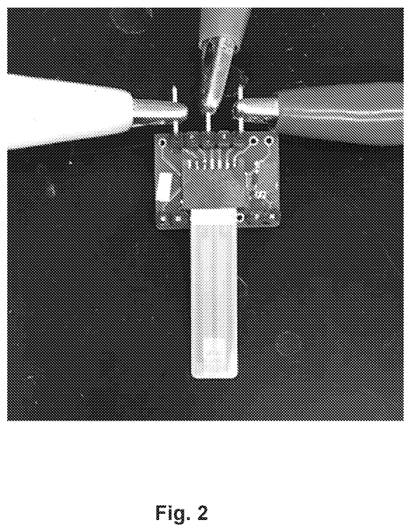

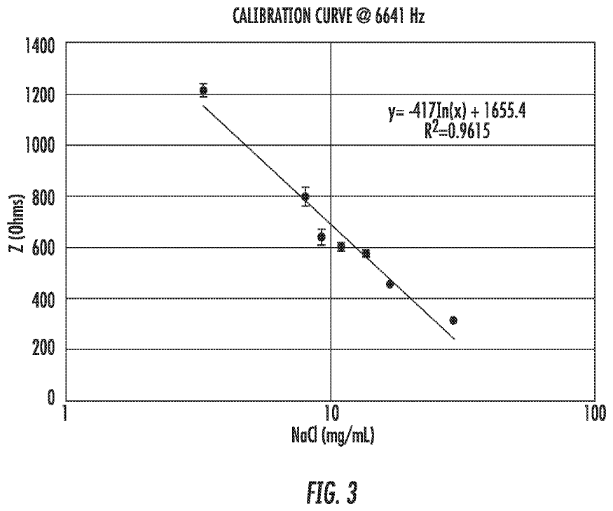

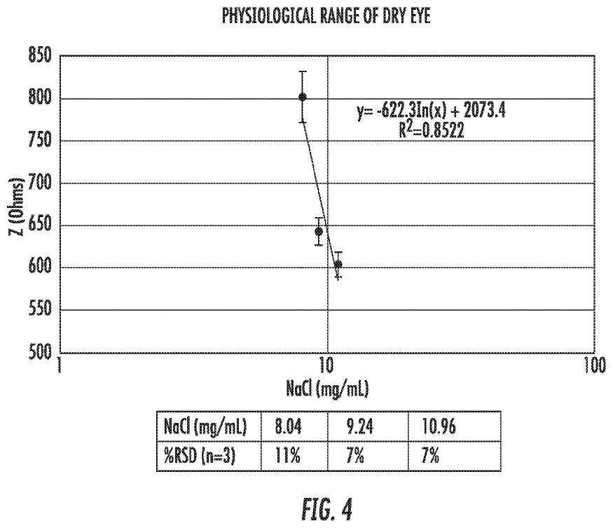

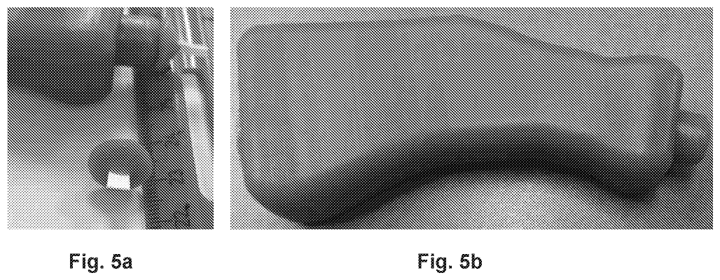

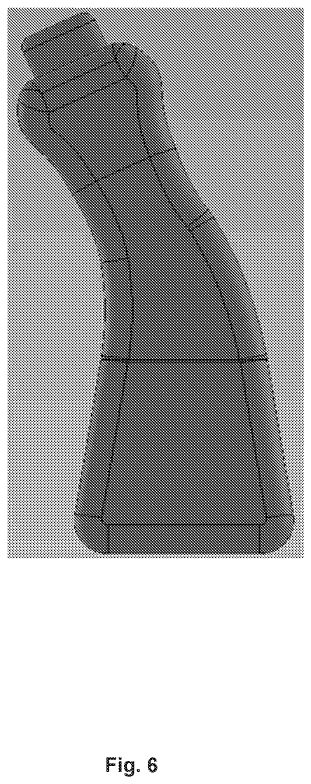

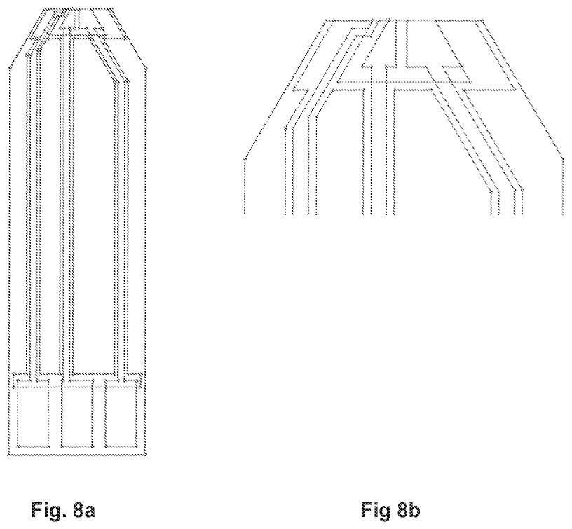

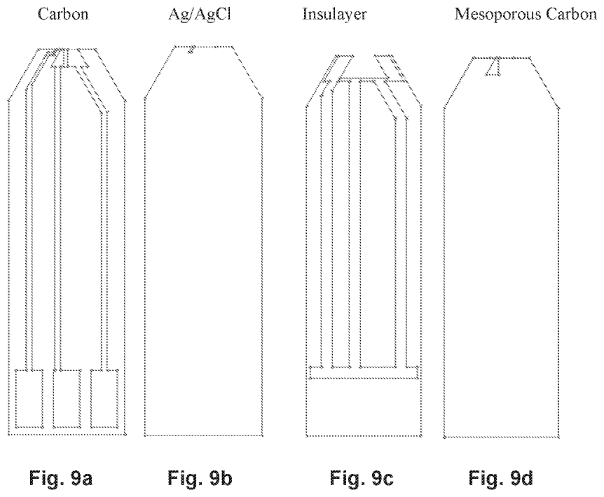

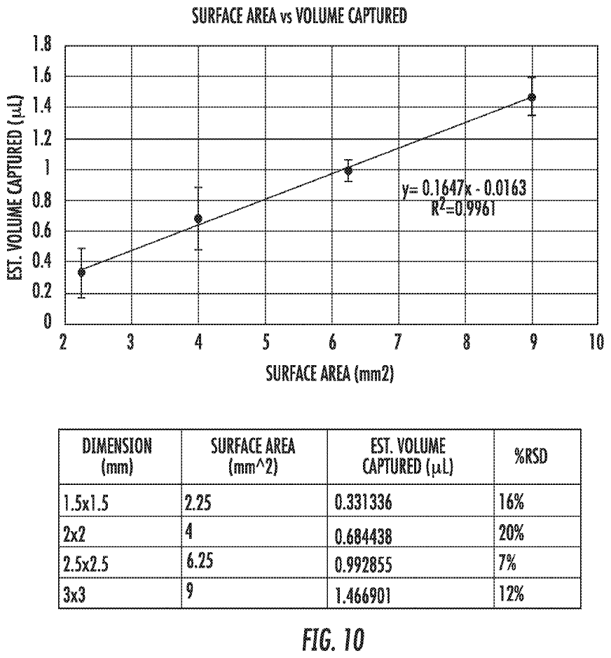

Embodiments herein relate to apparatus and methods for condition or disease detection. For example, turning to FIGS. 1-10, tear fluid can be drawn to a custom electrode from the eye using FDA approved filter paper. The measurement of osmolarity levels associated with dry eye can then be detected in the tear fluid using Electrochemical Impedance Spectroscopy (EIS) in a handheld point-of-care device. Depending on the dry eye assessment through measurement of ocular osmolarity, different treatment options may be indicated.

For example, treatment options may include punctal occlusion, meibomian gland therapy or some type of ocular medication (e.g., an anti-inflammatory topical). These medications include but are not limited to dozens of over-the-counter eye drops, or possibly prescription drugs like Restasis® (cyclosporine ophthalmic emulsion), Xiidra® (lifitegrast ophthalmic solution) or hydroxypropyl cellulose ophthalmic drops.

Further embodiments herein relate to the measurement of osmolarity or osmolality in fluids or secretions not in equilibrium with the extra-cellular fluids of the body, including but not limited to gastric juice, saliva and sweat. Serum osmolality is normally between 275 to 295 mOsm/kg; it increases with dehydration and decreases with over-hydration. Thus, a direct measurement of hydration levels is possible with measurement of serum osmolality. Dehydration can be treated with increased fluid intake, while increasing salts in the body (e.g., intravenously) can be used to treat over-hydration.

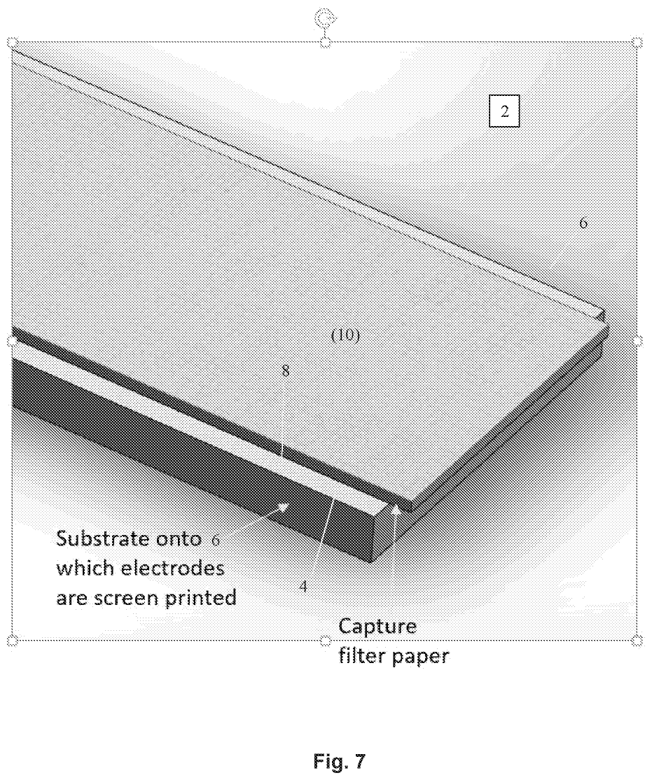

By way of example for embodiments that involve assessment of tear osmolarity, as shown in FIG. 7, a sensor strip 2 can be utilized. The sensor 2 may include PVC or similar substrate 4 and screen-printed electrode leads 6, which include dried reagents and protein (together, 8) for subsequent tear assay. Coupled to substrate 4 is an absorbent material, such as filter paper 10, to absorb tear fluid, with the shape and dimensions of filter paper determined based on absorption tests. For example, the filter paper may be 1.75 mm×1.75 mm. The dimensions of the electrodes, for example, a 3-lead electrode, are determined based on filter paper dimensions. Electrode materials may include, but are not limited to, carbon conductive ink, silver chloride ink, and novel mesoporous carbon ink and glue. Mesoporous ink facilitates electrochemical measurement and contains protein to detect the molecule of interest.

Turning to FIGS. 1-6 and 8-10, further examples of sensor embodiments and data are show. The sensor in some embodiments includes 4 layers of screen print inks, each with its own stencil. The four layers of ink are shown as separate stencil designs as they would be printed, the first layer being carbon, then Ag/AgCl, etc.

In all sensor embodiments, the sensor would be operably configured to utilize electrochemical impedance. For example, a power supply, computer/software, potentiostat, and/or further EIS components necessary for the sensor to operate/provide measurements are provided. Such EIS system components are available commercially through sources such as NuVant Systems.

### Example

In one embodiment, a sensor contains three electrodes (working, counter & reference). A 50% NaCl/50% ferricyanide [10 mM] (electron mediator) solution is applied to sensor and the applied voltage is −0.17 V with an amplitude of 5 mV and sweeping a range of frequencies from 1 to 100 k Hz. Resistance to electron flow to the sensor is measured at the range of frequencies. At the optimal frequency, the complex impedance can be correlated to the concentration of NaCl.

Advantageously, test results can obtained quickly (e.g., in under 90 seconds). Concentration measurements are taken at the optimal frequency of the system, providing the best resolution. Moreover, the system provides accurate readings over an extreme range of concentrations (3-30 mg/mL), and good results are obtained with a range of sample volumes between 5 and 100 uL, thereby indicating that the sensor is robust across volume changes. Furthermore, bare gold sensors may be used, without any sample preparation or employed filters on the sensor surface.

The results may be displayed on the device and/or an external device such as a phone or computer screen.

By way of further example, urinalysis through osmolarity measurement may provide useful assessment information:

Higher than Normal Measurements May Indicate:
Heart failure
Loss of body fluids (dehydration)
Narrowing of the kidney artery (renal artery stenosis)
Shock
Sugar (glucose) in the urine
Syndrome of inappropriate ADH secretion (SIADH)

Lower than Normal Measurements May Indicate:
Damage to kidney tubule cells (renal tubular necrosis)
Drinking too much fluid
Kidney failure
Severe kidney infection (pyelonephritis)

Thus, methods and devices disclosed herein may be useful in the measurement of osmolarity and osmolality of a variety of body fluids and for a variety of conditions or diseases.

The claims are not meant to be limited to the materials, methods, embodiments, and examples described herein.

What is claimed is:

1. A method for collecting and analyzing osmolarity in a bodily fluid, comprising:
contacting an absorbent material on a sensor with said bodily fluid, wherein said sensor comprises a substrate and an electrode operably configured to provide an electrochemical impedance measurement of said bodily fluid, and
measuring an electrochemical impedance of said bodily fluid to determine osmolarity.

2. The method of claim 1, wherein said bodily fluid is tear fluid.

3. The method of claim 2, further comprising detecting an indication of dry eye, wherein dry eye is indicated by a measured osmolarity range of between 290-316 mOsm/L.

4. The method of claim 3, further comprising treating dry eye based on said osmolarity.

5. The method of claim 4, wherein said treating includes one or more of punctal occlusion, meibomian gland therapy, an ocular anti-inflammatory medication, cyclosporine ophthalmic emulsion, lifitegrast ophthalmic solution, or hydroxypropyl cellulose ophthalmic drops.

6. The method of claim 1, wherein said fluid is salvia or urine.

7. A method for analyzing osmolality in a bodily fluid sample, comprising measuring an electrochemical impedance of said bodily fluid sample with a device to determine osmolality.

8. The method of claim 7, wherein said fluid is blood serum.

9. The method of claim 8, further comprising detecting an indication of dehydration or overhydartion, wherein serum osmolality is normally between 275 to 295 mOsm/kg, with a higher osmolality indicating dehydration and a lower osmolality indicating overhydration.

10. The method of claim 9, further comprising treating said dehydration or overhydration based on said osmolarity.

11. The method of claim 10, wherein said treating comprises increased fluid intake for dehydration or increasing salts in the body for over-hydration.
